(19) United States
(12) Patent Application Publication
(10) Pub. No.: US 2024/0116890 A1
(43) Pub. Date: Apr. 11, 2024

(54) LARGE SCALE SYNTHESIS OF CANNABINOL

(71) Applicant: Colorado Chromatograpy, LLC, Parker, CO (US)

(72) Inventors: Arianna C. COLLINS, Parker, CO (US); Ivan CRUCES, Parker, CO (US); Kyle RAY, Parker, CO (US); Westley CRUCES, Parker, CO (US)

(21) Appl. No.: 18/476,926

(22) Filed: Sep. 28, 2023

Related U.S. Application Data
(60) Provisional application No. 63/410,901, filed on Sep. 28, 2022.

(51) Int. Cl. C07D 311/80

(52) U.S. Cl. CPC C07D 311/80

(57) ABSTRACT

Methods for producing cannabinol, cannabivarin, or derivatives thereof are disclosed. The methods disclosed herein can be employed for large scale synthesis or semi-synthesis of the compounds, including multi-kilogram quantities of the compounds.

### CROSS-REFERENCE TO RELATED APPLICATIONS

This application claims the benefit of priority of U.S. Provisional Patent Application No. 63/410,901 filed Sep. 28, 2022, the contents of which are hereby incorporated by reference in their entirety.

### BACKGROUND OF THE INVENTION

### I. Field of the Invention

This invention relates to the fields of organic chemistry and medicinal chemistry.

### II. Background

Cannabinol (CBN) is one of the many products from the degradation of Tetrahydrocannabinol (THC), that has potential for use as a starting material into new and useful cannabinoids. While it exists naturally in hemp plants, the amount is low.

Large scale synthesis of CBN have been conducted via the dehydrogenation of THC (Δ9/Δ8) by excess sulfur leading to a very crude CBN product, which is difficult for future use as it leads to lower yields and poor conversions. Previous attempts at purification were conducted via functional group interconversion of the alcohol on the primary benzene ring (WO 2004/043946 A1), however such attempts were limited to sulfonyl esters and done to purify cannabinoids from non-cannabinoid products rather than crude reaction products. Further, previous attempts at purification were not performed on CBN synthesized from THC via excess sulfur, which creates harsh sulfur contaminants that are difficult to remove from the synthesis products. Therefore, new methods for large scale synthesis of CBN are needed.

### SUMMARY OF THE INVENTION

The present disclosure provides methods for the synthesis of cannabinol (CBN) or derivatives thereof. In some aspects, the CBN derivatives include alkyl tails, which may be branched, unbranched, cyclic, aromatic, and/or halogenated. Surprisingly, these methods, even when done on a large scale, have been found to lead to a more pure product than previous large scale synthesis schemes.

Some aspects of the disclosure are directed to a process for the preparation of a CBN, comprising providing a tetrahydrocannabinol (THC) derivative to a reaction vessel. Sulfur may be added to the reaction vessel to generate a crude cannabinol product. In some aspects, the crude cannabinol product is contacted with a protecting group compound to generate a protected-cannabinol. In some aspects, the protected-cannabinol is contacted with a metal hydroxide and an alcohol to generate a cannabinol of formula I:

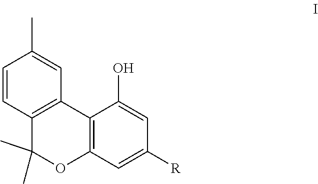

or a derivative thereof, wherein R comprises an alkyl group, a phenyl group, a benzyl group, a styryl group, a heterocycle, a fluorinated alkyl group, branched C₁-C₁₀ alkyl, unbranched C₁-C₁₀, branched C₁-C₁₀ alkenyl, or unbranched C₁-C₁₀ alkenyl; wherein said alkyl or alkenyl are optionally substituted with one or more hydroxyl, alkoxy, deuterium, halo (fluoro, chloro, bromo, iodo), thio, amino, or cyano groups. In some embodiments, the THC derivative is represented by formula II:

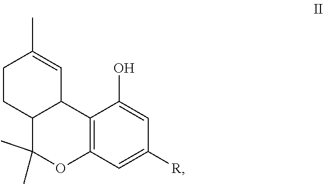

where R is as described above. In some embodiments, the protected-cannabinol is represented by formula III:

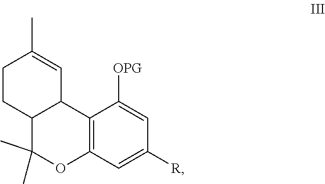

where PG is a protecting group and R is as described above.

In some aspects, sulfur is provided at 2-8 molar equivalents to the tetrahydrocannabinol or tetrahydrocannabinol derivative. In some embodiments, the sulfur is S₂, S₃, S₄, S₅, S₆, S₇, S₈, or any combination thereof. In further aspects, the reaction vessel is heated to a temperature sufficient to generate the crude cannabinol product. The reaction vessel can be heated to a temperature ranging from 180° C. to 260° C. In some embodiments, the reaction vessel is heated to a temperature of any one of, less than, greater than, or between 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259 or 260° C., or any range derivable therein. Certain aspects further comprise purifying the crude cannabinol product. In some aspects, the purifying comprises distillation or chromatography.

Certain aspects further comprise contacting the crude cannabinol product with the protecting group compound in the presence of a Lewis base, an alkyl amine, and/or hexanes. In some aspects, the Lewis base comprises 4-dimethylaminopyridine, imidazole, pyrrole, pyrrolidine, morpholine, or a combination thereof. In some aspects, the alkyl amine comprises N,N-Diisopropylethylamine, tert-butylamine, butylamine, methyl, ethylamine, pyridine, or a combination thereof. In certain aspects, the protecting group comprises acetyl, 2-nosyl, 3-nosyl, 4-nosyl, tosyl, benzenesulfonyl, 1-naphthyl, 2-naphthyl, adamantoyl, an aromatic ester, or an aromatic sulfonate. Acetyl, 2-nosyl, 3-nosyl, 4-nosyl, tosyl, benzenesulfonyl, and adamantoyl protecting groups can be installed upon reaction of the alcohol with the corresponding acid halide, for example. 1-Naphthyl and 2-naphthyl protecting groups can be installed upon reaction of the alcohol with the corresponding naphthyl halide, for example. An aromatic ester protecting group can be installed upon reaction of the alcohol with a corresponding aromatic acid halide, for example. An aromatic sulfonate protecting group can be installed upon reaction with a corresponding aromatic sulfonyl halide, for example.

In certain aspects, contacting the crude cannabinol product with the protecting group compound occurs at room temperature. Certain aspects further comprise purifying the protected-cannabinol. In some aspects the purifying of the protected-cannabinol comprises crystallization, distillation, and/or chromatography.

In certain aspects, the metal hydroxide is lithium hydroxide, sodium hydroxide, potassium hydroxide, rubidium hydroxide, cesium hydroxide, magnesium hydroxide, calcium hydroxide, strontium hydroxide, barium hydroxide, or a combination thereof. In certain aspects, contacting the protected-cannabinol with a metal hydroxide and an alcohol occurs at room temperature. In certain aspects, the alcohol comprises methanol, ethanol, isopropylalcohol, propylalcohol, butanol, sec-butanol, tert-butanol, trifluoroethanol, hexafluoroisopropanol, or any combination thereof.

Certain aspects of the disclosure concern any process herein, wherein the process further comprises contacting divarin or a derivative thereof with a terpene to generate cannabidivarin, or a derivative thereof. In some embodiments, the divarin derivative is represented by formula IV:

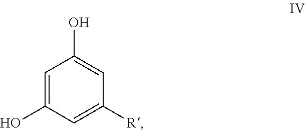

where R′ is an alkyl group. In some embodiments, the cannabidivarin derivative is prepresented by formula V:

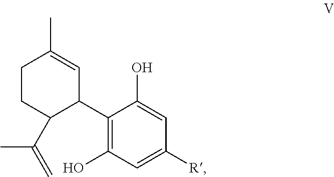

where R′ is an alkyl group. In some aspects, the cannabidivarin is contacted with an acid to generate the tetrahydrocannabivarin or derivative thereof. In some embodiments, the tetrahydrocannabivarin derivative is represented by formula VI:

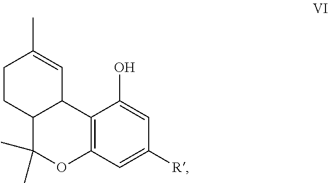

where R′ is an alkyl group. The terpene may comprise (1S,4R)-1-methyl-4-(prop-1-en-2-yl)cyclohex-2-enol and/or cis-Verbenol. In some aspects, the acid comprises a Bronsted acid and/or a Lewis acid. The acid may comprise p-toluenesulfonic acid, hydrochloric acid, camphorsulfonic acid, trifluoroacetic acid, trifluoromethanesulfonic acid, boron trifluoride etherate, triisobutlyaluminum (TIBA), triethylaluminium, indium (III) trifluoromethanesulfonate, scandium (III) trifluoromethanesulfonate, or a combination thereof. The tetrahydrocannabivarin or derivative thereof can be contacted with sulfur to generate the corresponding cannabivarin or derivative thereof. The cannabivarin or derivative hydroxyl group can be protected with any of the protecting groups described above and the protecting group can be subsequently removed with any of the metal hydroxide compounds described above. In some embodiments, the cannabivarin or derivative thereof is represented by formula VII:

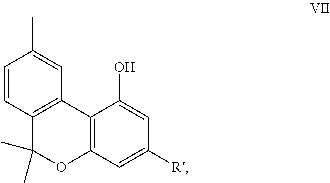

where R′ is as described above.

In certain aspects, the cannabinol derivative comprises cannabivarin. In certain aspects, the R′ group comprises a propyl group and/or a pentyl group.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Exemplary reaction scheme for access to CBN using sulfur.

FIGS. 2A-2B Exemplary reaction scheme. FIG. 2A shows access to CBN-Acetate by functional group interconversion. FIG. 2B is a ¹H NMR of the CBN acetate product.

FIGS. 3A-3B Exemplary reaction scheme. FIG. 3A shows access to CBN from CBN-acetate using functional group interconversion. FIG. 3B is a ¹H NMR of the CBN product.

FIGS. 4A-4C Exemplary reaction scheme. FIG. 4A shows access to CBV/CBNV. FIG. 4B is a ¹H NMR of the CBNV-acetate product. FIG. 4C is a ¹H NMR of the CBNV-acetate product.

### DETAILED DESCRIPTION OF THE INVENTION

Over 120 different phytocannabinoids have been isolated from Cannabis sativa. Of these, Δ9-tetrahydrocannabinol (Δ9-THC) and cannabidiol (CBD) are the most abundant and widely studied. The inventors have surprisingly discovered a method of synthesizing other CBN products or derivatives for further study. Certain aspects concern processes for generating CBN from CBD isolate to D8 THC in attempts to purify the CBN from the harsh sulfur contaminants found during vulcanization of sulfur. In some aspects, the process utilizes acid chlorides and acid anhydrides in addition or in substitution to sulfonyl chlorides.

In certain aspects, CBN products or derivatives, which may be made from aromatization of THC or derivatives thereof, are protected by converting an alcohol on the CBN product or derivative into a protected alcohol. The protection of the alcohol may be done by any method known in the art using any protecting group known in the art, including those disclosed herein. Additional examples of alcohol protecting groups, including phenolic protecting groups, can be found in Greene's Protective Groups in Organic Synthesis, Peter Wutts and Theeodora Greeene, John Wiley & Sons, 2007, the entirety of which is incorporated herein by reference. In some aspects, repeated crystallization of the protected compound leads to very pure protected CBN or protected CBN derivative. The protected CBN or derivative thereof may be 90%, 91%, 92%, 93%, 94%, 95%, 96%, 97%, 98%, 99%, 99.5%, 99.9%, or 100% pure. In some aspects the crude CBN can be isolated by distillation or chromatography, leading to a 90%, 91%, 92%, 93%, 94%, 95%, 96%, 97%, 98%, 99%, 99.5%, 99.9%, or 100% pure product.

In some aspects, the protected CBN or derivative, purified or unpurified, can be mixed with a metal hydroxide in the presence of an alcohol, leading to a deprotection reaction and leaving behind the CBN or derivative. Examples of CBN derivatives also applicable for this reaction scheme include cannabivarin (CBV/CBNV), or other alkyl side chains on the resorcinol moiety found on CBN. In certain aspects, the process can generate 1, 2, 3, 4, 5, 6, 7, 8, 9, 10, 11, 12, 13, 14, 15, 16, 17, 18, 19, 20, 21, 22, 23, 24, 25 kilograms of CBN, CBV/CBNV, or a derivative thereof.

### Chemical Definitions

The terms CBN and cannabinol are used interchangeably herein. The terms CBV, CBNV, and cannabivarin are used interchangeably herein. The phrase “semi-synthetic” is defined as a method that employs natural compounds or compounds derived from natural compounds as starting materials to produce different compounds.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, alkyl, halogenated alkyl, alkoxy, alkenyl, alkynyl, aryl, heteroaryl, aldehyde, amino, carboxylic acid, cyano, ester, ether, halide, hydroxy, ketone, nitro, silyl, sulfo-oxo, sulfonyl, sulfone, sulfoxide, and thiol. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” includes straight-chain alkyl, branched-chain alkyl, cycloalkyl (alicyclic), heteroatom-unsubstituted alkyl, heteroatom-substituted alkyl, heteroatom-unsubstituted Cₙ-alkyl, and heteroatom-substituted Cₙ-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), CH₂CH(CH₃)₂ (iso-butyl), —C(CH₃)₃ (tent-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, CH₂F, —CH₂Cl, —CH₂Br, piperidinyl, —CH₂OH, —CH₂OCH₃, —CH₂OCH₂CF₃, CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cn-alkenyl, and heteroatom-substituted Cn-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cn-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), CH₂CH═CHCH₃, —CH═CH—C₆H₅, —CH₂CHC(CH₃)₂ (isoprenyl), and CH₂CHC(CH₃)CH₂(CH₂CHC(CH₃)CH₂)₃CH₂CHC(CH₃)₂ (geranylfarnesyl). The term “heteroatom-substituted Cn-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, dihydrofuranyl, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, C₆H₄CH(CH₃)₂, —C₆H₄CH(CH₂)₂, —C₆H₃(CH₃)CH₂CH₃, —C₆H₄CH═CH₂, C₆H₄CH═CHCH₃, —C₆H₄CCH, —C₆H₄CCCH₃, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, C₆H₄NH₂, —C₆H₄NHCH₃, —C₆H₄N(CH₃)₂, —C₆H₄CH₂OH, —C₆H₄CH₂OC(O)CH₃, C₆H₄CH₂NH═, —C₆H₄CF₃, —C₆H₄CN, —C₆H₄CHO, —C₆H₄CHO, —C₆H₄C(O)CH₃, C₆H₄C(O)C₆H₅, —C₆H₄CO₂H, —C₆H₄CO₂CH₃, —C₆H₄CONH₂, —C₆H₄CONHCH₃, C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents.

The term “aralkyl” includes heteroatom-unsubstituted aralkyl, heteroatom-substituted aralkyl, heteroatom-unsubstituted Cn-aralkyl, heteroatom-substituted Cn-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cn-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C7-C10-aralkyl has 7 to 10 carbon atoms. Non-limiting examples of heteroatom-unsubstituted aralkyls are: phenylmethyl (benzyl, Bn) and phenylethyl. The term “heteroatom-substituted Cn-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cn-acyl, heteroatom-substituted Cn-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cn-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cn-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, CO₂H, —CO₂—, —CO₂CH₃, —CO₂CH₂CH₃, —CO₂CH₂CH₂CH₃, —CO₂CH(CH₃)₂, CO₂CH(CH₂)₂, —C(O)NH₂ (carbamoyl), —C(O)NHCH₃, —C(O)NHCH₂CH₃, CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cn-alkoxy, and heteroatom-substituted Cn-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cn-alkoxy” refers to a group, having the structure —OR, in which R is a heteroatom-unsubstituted Cn-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cn-alkoxy” refers to a group, having the structure —OR, in which R is a heteroatom-substituted Cn-alkyl, as that term is defined above. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cn-alkenyloxy, and heteroatom-substituted Cn-alkenyloxy. The term “heteroatom-unsubstituted Cn-alkenyloxy” refers to a group, having the structure —OR, in which R is a heteroatom-unsubstituted Cn-alkenyl, as that term is defined above. The term “heteroatom-substituted Cn-alkenyloxy” refers to a group, having the structure —OR, in which R is a heteroatom-substituted Cn-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cn-alkynyloxy, and heteroatom-substituted Cn-alkynyloxy. The term “heteroatom-unsubstituted Cn-alkynyloxy” refers to a group, having the structure —OR, in which R is a heteroatom-unsubstituted Cn-alkynyl, as that term is defined above. The term “heteroatom-substituted Cn-alkynyloxy” refers to a group, having the structure —OR, in which R is a heteroatom-substituted Cn-alkynyl, as that term is defined above.

The term “aryloxy” includes heteroatom-unsubstituted aryloxy, heteroatom-substituted aryloxy, heteroatom-unsubstituted Cn-aryloxy, heteroatom-substituted Cn-aryloxy, heteroaryloxy, and heterocyclic aryloxy groups. The term “heteroatom-unsubstituted Cn-aryloxy” refers to a group, having the structure —OAr, in which Ar is a heteroatom-unsubstituted Cn-aryl, as that term is defined above. A non-limiting example of a heteroatom-unsubstituted aryloxy group is —OC₆H₅. The term “heteroatom-substituted Cn-aryloxy” refers to a group, having the structure —OAr, in which Ar is a heteroatom-substituted Cn-aryl, as that term is defined above.

The term “aralkyloxy” includes heteroatom-unsubstituted aralkyloxy, heteroatom-substituted aralkyloxy, heteroatom-unsubstituted Cn-aralkyloxy, heteroatom-substituted Cn-aralkyloxy, heteroaralkyloxy, and heterocyclic aralkyloxy groups. The term “heteroatom-unsubstituted Cn-aralkyloxy” refers to a group, having the structure —OAr, in which Ar is a heteroatom-unsubstituted Cn-aralkyl, as that term is defined above. The term “heteroatom-substituted Cn-aralkyloxy” refers to a group, having the structure —OAr, in which Ar is a heteroatom-substituted Cn-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cn-acyloxy, heteroatom-substituted Cn-acyloxy, alkylcarbonyloxy, arylcarbonyloxy, alkoxycarbonyloxy, aryloxycarbonyloxy, and carboxylate groups. The term “heteroatom-unsubstituted Cn-acyloxy” refers to a group, having the structure —OAc, in which Ac is a heteroatom-unsubstituted Cn-acyl, as that term is defined above. For example, —OC(O)CH₃ is a non-limiting example of a heteroatom-unsubstituted acyloxy group. The term “heteroatom-substituted Cn-acyloxy” refers to a group, having the structure —OAc, in which Ac is a heteroatom-substituted Cn-acyl, as that term is defined above. For example, —OC(O)OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cn-alkylamino, and heteroatom-substituted Cn-alkylamino. The term “heteroatom-unsubstituted Cn-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cn-alkylamino” includes groups, having the structure —NHR, in which R is a heteroatom-unsubstituted Cn-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cn-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cn-alkylamino” includes groups, having the structure —NHR, in which R is a heteroatom-substituted Cn-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cn-alkenylamino, heteroatom-substituted Cn-alkenylamino, dialkenylamino, and alkyl(alkenyl)amino groups. The term “heteroatom-unsubstituted Cn-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cn-alkenylamino” includes groups, having the structure —NHR, in which R is a heteroatom-unsubstituted Cn-alkenyl, as that term is defined above. The term “heteroatom-substituted Cn-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cn-alkenylamino” includes groups, having the structure —NHR, in which R is a heteroatom-substituted Cn-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cn-alkynylamino, heteroatom-substituted Cn-alkynylamino, dialkynylamino, alkyl(alkynyl)amino, and alkenyl(alkynyl)amino groups. The term “heteroatom-unsubstituted Cn-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cn-alkynylamino” includes groups, having the structure —NHR, in which R is a heteroatom-unsubstituted Cn-alkynyl, as that term is defined above. The term “heteroatom-substituted Cn-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cn-alkynylamino” includes groups, having the structure —NHR, in which R is a heteroatom-substituted Cn-alkynyl, as that term is defined above.

The term “arylamino” includes heteroatom-unsubstituted arylamino, heteroatom-substituted arylamino, heteroatom-unsubstituted Cn-arylamino, heteroatom-substituted Cn-arylamino, heteroarylamino, heterocyclic arylamino, and alkyl(aryl)amino groups. The term “heteroatom-unsubstituted Cn-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cn-arylamino” includes groups, having the structure —NHR, in which R is a heteroatom-unsubstituted Cn-aryl, as that term is defined above. The term “heteroatom-substituted Cn-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cn-arylamino” includes groups, having the structure —NHR, in which R is a heteroatom-substituted Cn-aryl, as that term is defined above.

The term “aralkylamino” includes heteroatom-unsubstituted aralkylamino, heteroatom-substituted aralkylamino, heteroatom-unsubstituted Cn-aralkylamino, heteroatom-substituted Cn-aralkylamino, heteroaralkylamino, heterocyclic aralkylamino groups, and diaralkylamino groups. The term “heteroatom-unsubstituted Cn-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cn-aralkyl, as that term is defined above. The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cn-aralkylamino” includes groups, having the structure —NHR, in which R is a heteroatom-substituted Cn-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cn-amido, heteroatom-substituted Cn-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cn-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cn-amido” includes groups, having the structure NHR, in which R is a heteroatom-unsubstituted Cn-acyl, as that term is defined above. The group, —NHC(O)CH₃, is a non-limiting example of a heteroatom-unsubstituted amido group. The term “heteroatom-substituted Cn-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cn-amido” includes groups, having the structure —NHR, in which R is a heteroatom-unsubstituted Cn-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cn-alkylthio, and heteroatom-substituted Cn-alkylthio. The term “heteroatom-unsubstituted Cn-alkylthio” refers to a group, having the structure —SR, in which R is a heteroatom-unsubstituted Cn-alkyl, as that term is defined above. The group, —SCH₃, is an example of a heteroatom-unsubstituted alkylthio group. The term “heteroatom-substituted Cn-alkylthio” refers to a group, having the structure —SR, in which R is a heteroatom-substituted Cn-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cn-alkenylthio, and heteroatom-substituted Cn-alkenylthio. The term “heteroatom-unsubstituted Cn-alkenylthio” refers to a group, having the structure —SR, in which R is a heteroatom-unsubstituted Cn-alkenyl, as that term is defined above. The term “heteroatom-substituted Cn-alkenylthio” refers to a group, having the structure —SR, in which R is a heteroatom-substituted Cn-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cn-alkynylthio, and heteroatom-substituted Cn-alkynylthio. The term “heteroatom-unsubstituted Cn-alkynylthio” refers to a group, having the structure —SR, in which R is a heteroatom-unsubstituted Cn-alkynyl, as that term is defined above. The term “heteroatom-substituted Cn-alkynylthio” refers to a group, having the structure —SR, in which R is a heteroatom-substituted Cn-alkynyl, as that term is defined above.

The term “arylthio” includes heteroatom-unsubstituted arylthio, heteroatom-substituted arylthio, heteroatom-unsubstituted Cn-arylthio, heteroatom-substituted Cn-arylthio, heteroarylthio, and heterocyclic arylthio groups. The term “heteroatom-unsubstituted Cn-arylthio” refers to a group, having the structure —SAr, in which Ar is a heteroatom-unsubstituted Cn-aryl, as that term is defined above. The group, —SC₆H₅, is an example of a heteroatom-unsubstituted arylthio group. The term “heteroatom-substituted Cn-arylthio” refers to a group, having the structure —SAr, in which Ar is a heteroatom-substituted Cn-aryl, as that term is defined above.

The term “aralkylthio” includes heteroatom-unsubstituted aralkylthio, heteroatom-substituted aralkylthio, heteroatom-unsubstituted Cn-aralkylthio, heteroatom-substituted Cn-aralkylthio, heteroaralkylthio, and heterocyclic aralkylthio groups. The term “heteroatom-unsubstituted Cn-aralkylthio” refers to a group, having the structure —SAr, in which Ar is a heteroatom-unsubstituted Cn-aralkyl, as that term is defined above. The group, —SCH₂C₆H₅, is an example of a heteroatom-unsubstituted aralkyl group. The term “heteroatom-substituted Cn-aralkylthio” refers to a group, having the structure —SAr, in which Ar is a heteroatom-substituted Cn-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cn-acylthio, heteroatom-substituted Cn-acylthio, alkylcarbonyloxy, arylcarbonyloxy, alkoxycarbonyloxy, aryloxycarbonyloxy, and carboxylate groups. The term “heteroatom-unsubstituted Cn-acylthio” refers to a group, having the structure —SAc, in which Ac is a heteroatom-unsubstituted Cn-acyl, as that term is defined above. The group, —SCOCH₃, is an example of a heteroatom-unsubstituted acylthio group. The term “heteroatom-substituted Cn-acylthio” refers to a group, having the structure —SAc, in which Ac is a heteroatom-substituted Cn-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cn-alkylsilyl, and heteroatom-substituted Cn-alkylsilyl. The term “heteroatom-unsubstituted Cn-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cn-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cn-phosphonate, and heteroatom-substituted Cn-phosphonate. The term “heteroatom-unsubstituted Cn-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C₀-C₁₀-phosphonate has 0 to 10 carbon atoms. The groups, —P(O)(OH)₂, —P(O)(OH)OCH₃, —P(O)(OH)OCH₂CH₃, P(O)(OCH₃)₂, and —P(O)(OH)(OC₆H₅) are non-limiting examples of heteroatom-unsubstituted phosphonate groups. The term “heteroatom-substituted Cn-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C₀-C₁₀-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cn-phosphinate, and heteroatom-substituted Cn-phosphinate. The term “heteroatom-unsubstituted Cn-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C₀-C₁₀-phosphinate has 0 to 10 carbon atoms. The groups, —P(O)(OH)H, —P(O)(OH)CH₃, —P(O)(OH)CH₂CH₃, P(O)(OCH₃)CH₃, and —P(O )(OC₆H₅)H are non-limiting examples of heteroatom-unsubstituted phosphinate groups. The term “heteroatom-substituted Cn-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C₀-C₁₀-phosphinate has 0 to 10 carbon atoms.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of Crude CBN

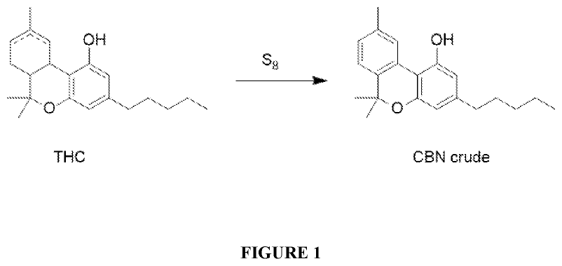

A mix of D8/D9 THC (12 kg) was added to a reactor equipped with an acid trap filled with metal hydroxide dissolved in water. Sulfur (2-8 equiv.) was added and the reaction is run neat from 180-260° C. until complete by HPLC (5-24 h), FIG. 1. The crude mixture is purified by distillation or taken forwards for functional group interconversion.

### Example 2

### Access to CBN-Acetate Using Functional Group Interconversion

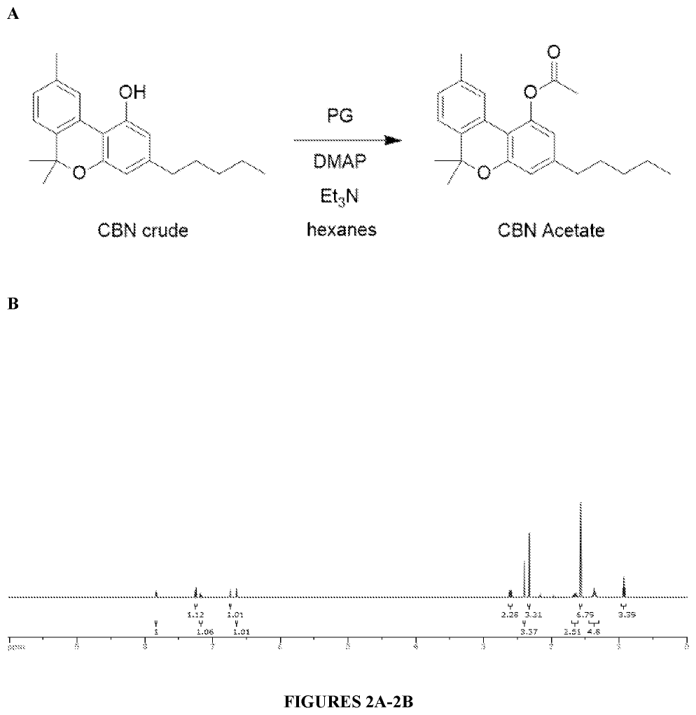

The crude CBN from Example 1 was dissolved in hexanes and added to a 200 L reactor. DMAP was added to the reactor. Afterwards, triethylamine was added slowly to the reactor. An electrophile/protecting group compound was added to an addition funnel and was added dropwise over 2 hours while the reaction mixture stirred at room temperature until complete by HPLC, FIG. 2A. Upon completion, the reaction mixture was quenched with 1M HCl, followed by bicarbonate and brine washes. The organic layer is kept and the water layer is discarded. The washed reaction mixture was then repeatedly crystallized at −20° C. in hexanes and limonene. The final product, CBN-Acetate, is a white crystalline powder, FIG. 2B ¹H NMR (500 MHz, CD₃CN) δ: 7.24 (1H), 6.73 (1H), 6.64 (1H), 2.60 (2H), 2.39 (3H), 2.32 (3H), 1.56 (7H), 1.36 (5H), 0.92 (3H).

### Example 3

### Access to CBN From CBN-Acetate Using Functional Group Interconversion

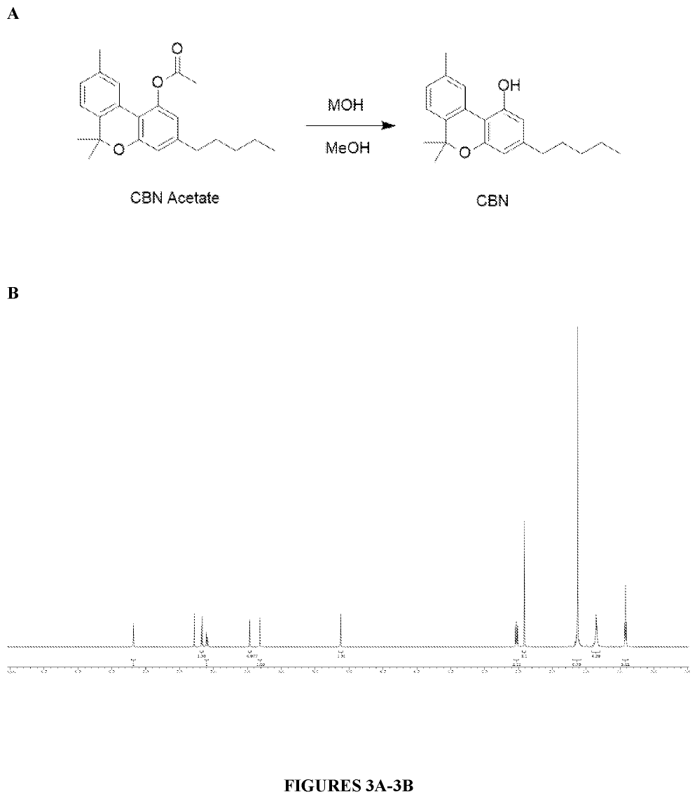

CBN-acetate was added to a 200 L reactor. MeOH was added. A solution of metal hydroxide (MOH) was added dropwise using an addition funnel. The reaction is stirred at room temperature and monitored using HPLC, FIG. 3A. Upon completion, the MeOH is evaporated and ethyl acetate is introduced to the reaction mixture. The reaction mixture is then washed with a solution of brine repeatedly until the solution reaches pH 7. The organic layer is kept and the water layer is discarded. The organic layer was then concentrated in vacuo. The final product CBN can be purified via distillation or chromatography. CBN is a white crystalline powder, FIG. 3B ¹H NMR (500 MHz, CDCl₃) δ: 7.09 (1H), 6.46 (1H), 6.31 (1H), 5.12 (1H), 2.52 (2H), 2.41 (3H), 1.63 (9H), 1.35 (4H), 0.91 (3H).

### Example 4

### Access to CBV/CBNV

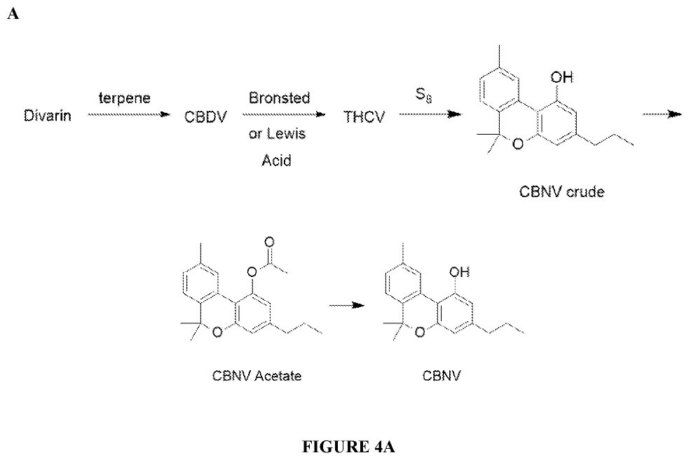

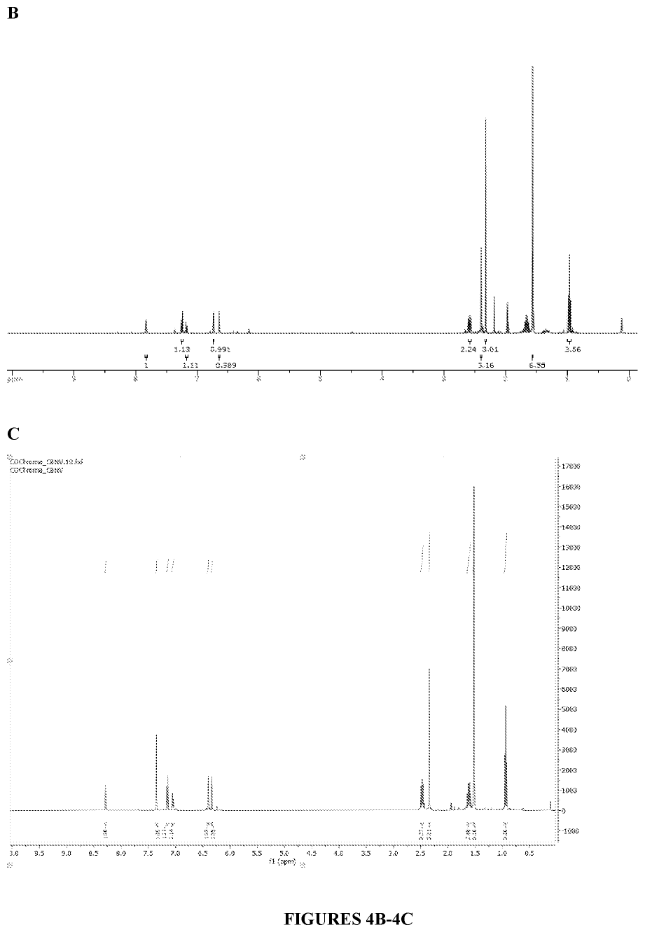

To a flask, (1S ,4R)-1-methyl-4-(prop-1-en-2-yl)cyclohex-2-enol and divarin were dissolved in dichloromethane and treated with acid and was stirred until complete by HPLC. The crude CBDV mixture is washed with aqueous bicarbonate and brine. It is then concentrated in vacuo. The crude CBDV is then redissolved in solvent (DCM, hexanes, or toluene). Acid is added and the reaction is stirred until complete by HPLC. The crude THCV mixture was washed with aqueous bicarbonate and brine. It is then concentrated in vacuo. The crude THCV is then purified by distillation or chromatography. A mixture of D8/D9 THCV is then added to a flask equipped with an acid trap filled with metal hydroxide dissolved in water. Sulfur (2-8 equiv.) was added and the reaction is run neat from 180-260° C. until complete by HPLC (1-48 h), FIG. 4A. The crude mixture is purified by distillation or taken forwards for functional group interconversion similar to Examples 2 and 3. CBV/CBNV-Acetate, is an orange oil, FIG. 4B ¹H NMR (500 MHz, CD₃CN) δ: 7.82 (1H), 7.24 (1H), 7.17 (1H), 6.73 (1H), 6.64 (1H), 2.58 (2H), 2.39 (2H), 1.58 (6H), 0.96 (3H) CBV/CBNV is a brown oil, FIG. 4C ¹H NMR (500 MHz, CDCl₃) δ: 8.29 (1H), 7.35 (1H), 7.15 (1H), 7.05 (1H), 6.40 (1H), 6.34 (1H), 2.48 (2H), 2.34 (3H), 1.61 (2H), 1.52 (6H), 0.94 (3H).

What is claimed is:

1. A process for the preparation of cannabinol or derivative thereof, the process comprising:
providing tetrahydrocannabinol or a derivative thereof to a reaction vessel;
providing sulfur to the reaction vessel to generate a crude cannabinol product;
contacting the crude cannabinol product with a protecting group compound to generate a protected-cannabinol; and
contacting the protected-cannabinol with a metal hydroxide and an alcohol to generate a cannabinol of formula I 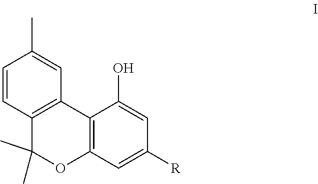
or a derivative thereof, wherein R comprises an alkyl group, a phenyl group, a benzyl group, a styryl group, a heterocycle, a fluorinated alkyl group, branched C₁-C₁₀ alkyl, unbranched C₁-C₁₀, branched C₁-C₁₀ alkenyl, or unbranched C₁-C₁₀ alkenyl; wherein said alkyl or alkenyl are optionally substituted with one or more hydroxyl, alkoxy, deuterium, halo, thio, amino, or cyano groups.

2. The process of claim 1, wherein sulfur is provided at 2-8 molar equivalents to the tetrahydrocannabinol or tetrahydrocannabinol derivative.

3. The process of claim 1, wherein the reaction vessel is heated to a temperature sufficient to generate the crude cannabinol product.

4. The process of claim 3, wherein the reaction vessel is heated to a temperature ranging from 180° C. to 260° C.

5. The process of claim 1, further comprising purifying the crude cannabinol product.

6. The process of claim 5, wherein the purifying comprises distillation or chromatography.

7. The process of claim 1, further comprising contacting the crude cannabinol product with the protecting group compound in the presence of a Lewis base, an alkyl amine, and hexanes.

8. The process of claim 7, wherein the Lewis base comprises 4-dimethylaminopyridine, imidazole, pyrrole, pyrrolidine, morpholine, or a combination thereof.

9. The process of claim 7, wherein the alkyl amine comprises N,N-diisopropylethylamine, tert-butylamine, butylamine, methyl, ethylamine, pyridine, or a combination thereof.

10. The process of claim 1, wherein a protecting group comprises acetyl, 2-nosyl, 3-nosyl, 4-nosyl, tosyl, benzenesulfonyl, 1-naphthyl, 2-naphthyl, adamantoyl, an aromatic ester, or an aromatic sulfonate.

11. The process of claim 1, wherein the alcohol comprises methanol, ethanol, isopropylalcohol, propylalcohol, butanol, sec-butanol, tert-butanol, trifluoroethanol, hexafluoroisopropanol.

12. The process of claim 11, wherein contacting the crude cannabinol product with the protecting group compound occurs at room temperature.

13. The process of claim 1, further comprising purifying the protected-cannabinol by crystallization, distillation, and/or chromatography.

14. The process of claim 1, wherein the metal hydroxide is lithium hydroxide, sodium hydroxide, potassium hydroxide, rubidium hydroxide, cesium hydroxide, magnesium hydroxide, calcium hydroxide, strontium hydroxide, barium hydroxide, or a combination thereof.

15. The process of claim 1, wherein contacting the protected-cannabinol with a metal hydroxide and an alcohol occurs at room temperature.

16. The process of claims 1, further comprising:
contacting divarin or derivative thereof with a terpene to generate a cannabidivarin; and
contacting the cannabidivarin with an acid to generate the tetrahydrocannabivarin or a derivative thereof.

17. The process of claim 16, wherein the terpene comprises (1S,4R)-1-methyl-4-(prop-1-en-2-yl)cyclohex-2-enol and/or cis-Verbenol.

18. The process of claim 16, wherein the acid comprises a Bronsted acid or a Lewis acid.

19. The process of any claim 16, wherein the acid comprises p-toluenesulfonic acid, hydrochloric acid, camphorsulfonic acid, trifluoroacetic acid, trifluoromethanesulfonic acid, boron trifluoride etherate, triisobutlyaluminum (TIBA), triethylaluminium, indium (III) trifluoromethanesulfonate, scandium (III) trifluoromethanesulfonate, or a combination thereof.

20. The process of claim 1, wherein the cannabinol derivative comprises cannabivarin.
